(19) United States
(12) Patent Application Publication
(10) Pub. No.: US 2024/0423839 A1
(43) Pub. Date: Dec. 26, 2024

(54) MEMBRANE REMOVAL DEVICE

(71) Applicant: Alcon Inc., Fribourg (CH)

(72) Inventors: Reto Grüebler, Greifensee (CH); Mikhail Ovchinnikov, Dana Point, CA (US)

(21) Appl. No.: 18/746,527

(22) Filed: Jun. 18, 2024

Related U.S. Application Data
(60) Provisional application No. 63/509,573, filed on Jun. 22, 2023.

(51) Int. Cl. A61F 9/008; A61B 18/00; A61B 18/22; A61B 90/00

(52) U.S. Cl. CPC A61F 9/00825; A61B 2018/00601; A61B 2018/2272; A61B 2090/0817; A61B 2218/007; A61F 2009/00863

(57) ABSTRACT

The present disclosure relates generally to surgical instruments, and more specifically, to ophthalmic surgical instruments and methods of making the same. In particular, a surgical instrument is provided. The surgical instrument includes a cannula. A spatula is formed at an end of the cannula. The spatula extends from the cannula at a non-zero angle relative to an exterior surface of the cannula. The spatula at least partially defines an aperture at the end of the cannula. An optical fiber extends along an interior of the cannula. The optical fiber is configured to direct laser light toward the spatula.

### BACKGROUND

Many microsurgical procedures may require precision cutting and/or removal of various body tissues. For example, vitreoretinal procedures such as retinotomies, retinectomies, autologous retinal transplants, membrane peels, and vitrectomies typically require the cutting, removal, dissection, delamination, coagulation, and/or manipulation of intraocular tissues such as the retina, vitreous humor, traction bands, and other ocular membranes.

The retina, or the innermost, light-sensitive layer lining the back wall of the eye, is responsible for receiving, modulating, and transmitting visual stimuli from the external environment to the optic nerve, and ultimately, the visual cortex of the brain. Structurally, the retina is a complex and delicate tissue with numerous types of cells arranged in multiple cellular layers or membranes. Due to the retina's role in vision and its fragility, damage thereto may result in severe loss of vision or even permanent blindness. Therefore, cutting, removal, or manipulation of the retina, as well as membranes or tissues attached thereto, must be done with great care to avoid unwanted retinal trauma.

Accordingly, there is a need in the art for improved surgical instruments for cutting, removal, and/or manipulation of the retina and membranes or tissues attached thereto.

### SUMMARY

The present disclosure relates generally to surgical instruments, and more specifically, to ophthalmic surgical instruments and methods of making the same.

In certain embodiments, a surgical instrument is provided. The surgical instrument includes a cannula. A spatula is formed at an end of the cannula. The spatula extends from the cannula at a non-zero angle relative to an exterior surface of the cannula. The spatula at least partially defines an aperture at the end of the cannula. An optical fiber extends along an interior or exterior of the cannula. The optical fiber is configured to direct laser light toward the spatula.

The following description and the related drawings set forth in detail certain illustrative features of one or more embodiments, including those described above.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an exemplary surgical instrument for use during an ophthalmic surgical procedure, in accordance with certain embodiments of the present disclosure.

FIGS. 2A-D illustrate cross-sectional views of examples of the cannula of FIG. 1, in accordance with certain embodiments of the present disclosure.

FIGS. 3A-C illustrate perspective views of examples of spatula geometries, in accordance with certain embodiments of the present disclosure.

FIG. 4 illustrates a cross-sectional view of the cannula engaged with a membrane of an eye, in accordance with certain embodiments of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

As used herein, the term “about” may refer to a +/−10% variation from the nominal value. It is to be understood that such a variation can be included in any value provided herein.

Particular embodiments disclosed herein provide surgical instruments for cutting and removing tissues from a surgical site, and more particularly, surgical instruments for cutting and removing membranes adjacent to the retina.

Various conditions affecting the retina may be treated by the cutting and removal of a membrane formed over or adjacent to the retina. For example, an epiretinal membrane (ERM) is a thin layer of scar tissue that can form at the vitreoretinal interface on the inner surface of the retina. ERMs are typically localized over the macula—the light-sensitive tissue at the center of the retina that is responsible for detailed vision. This scar tissue (membrane) may cause varying degrees of distortion and blurring of a patient's central field of vision, and thus, treatment thereof typically requires the removal of the membrane. ERMs can be either idiopathic (primary) or secondary. In idiopathic ERMs, cell proliferation may appear following posterior vitreous detachment (PVD) and a break in the internal limiting membrane (ILM). Secondary ERMs, on the other hand, may result from an already existing ocular pathology, such as proliferative vitreoretinopathy (PVR), proliferative diabetic retinopathy (PDR), hypertensive retinopathy, intraocular inflammation, occlusion of a retinal vein, retinal detachment (RD), and ocular trauma.

Currently, various microsurgical devices may be used to cut and remove ERMs from a patient's retina. Such devices include vitreous cutters (or vitrectomy probes), scissors, forceps, and surgical knives. However, utilization of such instruments may induce unwanted traction on the retina adjacent to a target membrane cut site, which can lead to retinal tears and even retinal detachments, or may require the use of both of a surgeon's hands. Additionally, in order to use a vitreous cutter to cut and remove an epiretinal membrane, a gap or tear must already exist between the epiretinal membrane and the retina.

Embodiments of surgical instruments described herein advantageously address the deficiencies of certain existing instruments and reduce the risk of unwanted damage to peripheral retinal tissues when cutting and/or removing ERMs and other membranes by providing a cannula with a spatula formed at an end of the cannula to facilitate mechanical lifting/separating of a membrane at a target surgical site, as well as an optical fiber to direct a laser beam to the lifted/separated membrane to cut the membrane with reduced risk of damage to the peripheral tissues.

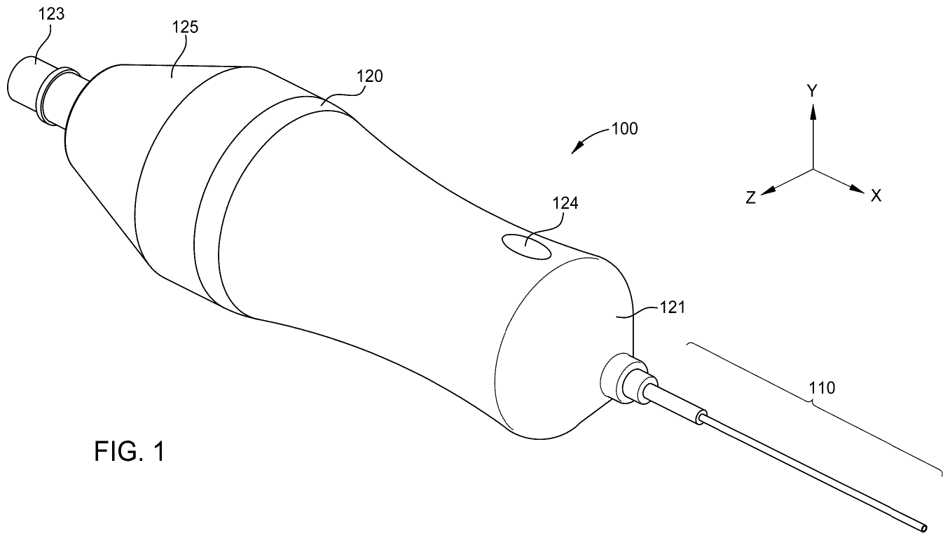

FIG. 1 illustrates a perspective view of an exemplary surgical instrument 100, according to certain embodiments described herein. As depicted in FIG. 1, the surgical instrument 100 comprises a cannula 110 and a base unit 120. The cannula 110 is partially and longitudinally disposed through a distal end 121 of the base unit 120 and may be directly or indirectly attached thereto within an interior chamber of the base unit 120. Note that, as described herein, a distal end or portion of a component refers to the end or the portion that is closer to a patient's body during use thereof. Additionally, a proximal end or portion of the component refers to the end or the portion that is distanced further away from the patient's body.

In some embodiments, the base unit 120 is a hand piece having an outer surface configured to be held by a user, such as a surgeon. For example, the base unit 120 may be ergonomically contoured to substantially fit the hand of the user. In some embodiments, the outer surface may be textured or have one or more gripping features formed thereon, such as one or more grooves and/or ridges. The base unit 120 may be made from any materials commonly used for such instruments and suitable for ophthalmic surgery. For example, the base unit 120 may be formed of a lightweight aluminum, stainless steel, a thermoplastic polymer, or other suitable material. In some embodiments, the base unit 120 is configured to be sterilized and used in more than one surgical procedure, or the base unit 120 may be a single-use device.

In some embodiments, the base unit 120 may include a control interface 124. The control interface 124 may include a button, slider, switch, haptic feedback, conductive element, touch element, or the like to facilitate detection of an input at the base unit 120. While the control interface 124 is shown as being proximate to the distal end 121 of the base unit 120, the control interface 124 may be positioned at other locations and with other orientations along a length of the base unit 120.

The base unit 120 further provides one or more ports 123 (e.g., one port 123 is depicted in FIG. 1) at a proximal end 125 thereof for one or more supply lines to be routed into an interior chamber of the base unit 120. For example, the port 123 may provide a connection between the base unit 120 and a vacuum line of a vacuum source for suction/aspiration. The port 123 may also provide a conduit for an optical fiber that extends between the cannula 110 and one or more light sources for providing laser light to the cannula 110. In some embodiments, the optical fiber may extend from the port 123 within an optical fiber cable configured to sheath the optical fiber form an external environment and facilitate coupling of the optical fiber to a light source and/or surgical console within which a light source may be integrated.

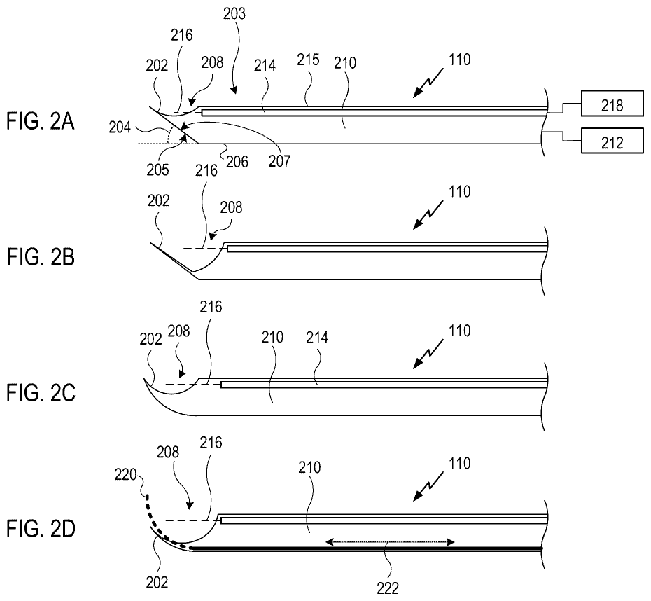

FIGS. 2A-D illustrate longitudinal cross-sectional views of examples of the cannula 110 of FIG. 1, in accordance with certain embodiments of the present disclosure. As shown, the cannula 110 may generally comprise an elongated hollow structure. For example, in some embodiments, the cannula 110 may have a cylindrical geometry. In other embodiments, however, the cannula 110 may have a non-cylindrical geometry (e.g., have a lateral cross-section comprising an oval, triangular, rectangular, etc. shape). The cannula 110 may be completely or partially formed of stainless steel, nickel titanium alloy, aluminum, or other metal or combination of metals. In some embodiments, the cannula 110 may be at least partially formed of a thermoplastic polymer. In some embodiments, the cannula 110 may include a coating, deposition, or other surface treatment. For example, at least a portion of the cannula 110 may have a roughened or laser-etched surface. Generally, the cannula 110 has a gauge size of twenty-nine, twenty-seven, twenty-five, twenty-three, or the like. However, other gauges and/or sizes are also contemplated.

In FIG. 2A, the cannula 110 is shown with a spatula 202 formed at a distal end 203 of the cannula 110. In some embodiments, the spatula 202 may be monolithic to the cannula 110. In other words, the spatula 202 may be formed by modifying a material of the cannula 110 at the distal end 203 to change a geometry of the cannula 110 at the distal end 203. For example, the distal end 203 of the cannula 110 may be bent, press-fit, or otherwise manipulated via a non-additive process to form the spatula 202. In some embodiments, the spatula 202 may be coupled to the cannula 110 through an additive process.

The spatula 202 facilitates the manipulation of materials, such as ocular tissues. In some embodiments, the spatula 202 may be utilized to delaminate a retinal membrane from a retina. The spatula 202 may have various geometries. For example, in some embodiments, the spatula 202 may extend from the cannula 110 at a non-zero angle 204 relative to an exterior surface 206 of the cannula 110. In some embodiments, the non-zero angle 204 is between thirty degrees and eighty degrees. In other embodiments, the non-zero angle 204 is less than thirty degrees, or more than eighty degrees. While the spatula 202 is shown as having a flat (e.g., planar) or substantially flat geometry wherein a distal surface 205 of the spatula 202 forms a consistent relative angle 204, other embodiments may include geometries which are variable in angle 204 relative to the exterior surface 206 of the cannula 110. For example, a curved or progressive geometry such as that shown in FIG. 2C is also contemplated. In some embodiments, the distal surface 205 may have a surface texture, treatment, or feature. For example, the distal surface 205 may be coated, infused, polished, etched, roughened, patterned, or the like. The surface texture, treatment, or feature on the distal surface 205 may facilitate increased or reduced traction for the spatula 202 when manipulating a membrane or other structure at a surgical site or environment.

In some embodiments, an aperture 208 is formed in the cannula 110 adjacent and proximal to the spatula 202. The aperture facilitates ingress of material to an interior 210 of the cannula 110. For example, the aperture 208 may be sized to allow for aspiration of material by application of negative pressure or vacuum to draw material through the aperture 208 and into the interior 210 of the cannula 110. In some embodiments, a portion of a periphery of the aperture 208 corresponds with an outside edge of the spatula 202. In other words, the aperture 208 may be at least partially defined by an edge of the spatula 202.

In some embodiments, a vacuum source 212 is in fluid communication with the cannula 110, via the port 123 of base unit 120, to apply a negative pressure or vacuum to the cannula 110 to aspirate material into the interior of the cannula 110 through the aperture 208. In such embodiments, the interior 210 of the cannula 110 may be configured to allow aspirated material to pass freely along the interior 210 of the cannula 110 and into, e.g., the base unit 120. The vacuum source 212 may be a standalone vacuum source or may be integrated with or operably coupled with a surgical console.

In some embodiments, an optical fiber 214 extends along the interior 210 of the cannula 110. In other embodiments, the optical fiber 214 extends along an exterior of the cannula 110. The optical fiber 214 may be configured to direct laser light 216 toward a proximal surface 207 of the spatula 202. The optical fiber 214 may be secured to a sidewall 215 on the interior 210 or exterior of the cannula 110, or the optical fiber 214 may be unsecured within the interior 210 of the cannula 110. For example, in some embodiments, the optical fiber 214 may be secured to the sidewall 215 via an adhesive. In other embodiments, the optical fiber 214 may be disposed within a rigid or semi-rigid sleeve extending along the interior 210, which may contact, or not contact, the sidewall 215.

In some embodiments, the spatula 202 and/or a portion of the spatula 202 may be aligned with the optical fiber 214 to allow for laser light 216 to be directed to the spatula 202 during use. In such embodiments, cavitation forces, heating, and other effects from the laser light 216 may be contained or otherwise shielded from causing damage to surrounding tissue or other structures by the spatula 202. For example, the positioning of the spatula 202 may reduce damage to surrounding retinal tissues when removing a retinal membrane.

The optical fiber 214 may include a cladding or may be unclad. In some embodiments, the optical fiber 214 is a sapphire fiber or other optically transmissive material. In some embodiments, the optical fiber 214 may have a uniform material composition along a length of the optical fiber 214. In other embodiments, the optical fiber 214 may have a first region having a first material composition and a second region having a second material composition where the first material composition and the second material composition are distinct from one another in one or more aspects. In some embodiments, the optical fiber 214 may be a single-core fiber. In other embodiments, the optical fiber 214 may be a multi-core fiber.

In some embodiments, a light source 218 is in optical communication with the optical fiber 214, via the port 123 of base unit 120, to provide light energy, as laser light 216, into the optical fiber 214 for propagation to the spatula 202. The light source 218 may be a standalone light source or may be integrated with or operably coupled with a surgical console. The light source 218 may be configured to generate a single type of light energy or multiple types of light energy. In some examples, the light source 218 may generate laser light 216 having a pulse rate within a range of about 100 hertz (Hz) and 10 kilohertz (kHz). In some embodiments, the laser source 218 may generate and propagate laser light 216 having a pulse rate within a range of about 10 kilohertz (kHz) and about 500 kHz, or between about 1 kHz and about 1500 Hz. In some embodiments, the laser source 218 may generate laser light 216 having a frequency of between approximately 100 Hz to approximately 5 kHz. In other embodiments, the laser source 218 may generate laser light 216 having a frequency of between approximately 100 Hz to approximately 1 kHz. In some embodiments, the laser source 218 may generate laser light 216 having a frequency of between approximately 100 Hz and approximately 500 Hz. Other pulse rate ranges are contemplated as well. In some examples, the laser source 218 produces a nanosecond, a picosecond, or a femtosecond first laser beam 104.

In some embodiments, the light source 218 may be configured to generate a treatment laser light 216 configured to ablate, sever, and/or remove material at the spatula 202. Again, the spatula 202 and/or a portion of the spatula 202 may be aligned with (e.g., disposed directly downstream of or distal to a distal end of) the optical fiber 214 to prevent cavitation forces, heating, and other effects from the treatment laser light 216 from causing damage to tissues, such as ocular tissues, adjacent to the distal end 203 of the cannula. Accordingly, the spatula 202 may serve as a “backstop” for the treatment laser light 216 distally propagated by optical fiber 214. In some embodiments, the proximal surface 207 of the spatula 202 may be patterned or roughened to either absorb or scatter the treatment laser light 216. For example, the proximal surface 207 may comprise a laser-etched pattern configured to provide maximum scattering of the treatment laser light 216 and prevent a bulk of the propagated treatment laser light 216 from being directed to any one region or tissue.

In some embodiments, the light source 218 may be configured to also (e.g., in addition to treatment laser light 216) generate a non-treatment laser light (e.g., aiming laser, illumination light, etc.) that produces little to no change in the material at the spatula 202 but may assist in illumination, tracking, aiming, etc. In such embodiments, the light source 218 may be configured to provide one or more different types of light energy to different cores of the optical fiber 214 or may be configured to provide one or more different types of light energy to a single core of the optical fiber 214.

FIG. 2B illustrates another example of the cannula 110. In the illustrated embodiment, the aperture 208 is larger and more open than the aperture 208 of the cannula 110 shown in FIG. 2A. In other words, the aperture 208 may extend more proximally along the length of the cannula 110 and/or may extend more radially around the cannula 110 toward a base of the spatula 202. In some embodiments, the curved shape of the aperture 208 may impact the shape and utility of the spatula 202. For example, in the illustrated embodiment, the spatula 202 may be more elongated to allow for engagement and manipulation of a thicker material (e.g., a membrane on the retina) than may the cannula 110 of FIG. 2A. In some embodiments, a more open aperture 208 may facilitate a broader geometry to the spatula 202 which may reduce force concentrations and allow for a more gentle material separation to avoid unwanted tearing or shearing at the surgical site.

FIG. 2C illustrates yet another example of the cannula 110 in which the spatula 202 has a curved geometry. In some embodiments, a radius of curvature of the spatula 202 may be uniform along a longitudinal length of the spatula 202, or the radius of curvature may vary along the length of the spatula 202. In the illustrated embodiment of FIG. 2C, the optical fiber 214 is further recessed away from the aperture 208 within the interior 210. While relative proximities are shown in the examples of FIGS. 2A-D, it is contemplated that the optical fiber 214 may be positioned to extend to different positions relative to the aperture 208. For example, the optical fiber 214 may terminate proximal to a proximal edge of the aperture 208, may terminate at the proximal edge of the aperture 208, or may extend distally beyond the proximal edge of the aperture 208 and into the aperture 208. In some embodiments, multiple optical fibers 214 or fiber cores may be included which terminate at different positions relative to the aperture 208.

FIG. 2D illustrates yet another example of the cannula 110. In the illustrated embodiment, an extendible tongue 220 is disposed within the interior 210 of the cannula 110. The extendible tongue 220 may be configured to extend along the spatula 202 to provide additional reach or facilitate mechanical manipulation of a material at the spatula 202. In some embodiments, the extendible tongue 220 is translatable in a longitudinal direction 222 by a user. For example, a user may operate a slide, trigger, or other control (e.g., control interface 124 of FIG. 1) on the base unit 120 to extend or retract the tongue 220 into/from the cannula 110. In some embodiments, the tongue 220 is flexible along a length of the tongue. In other embodiments, the tongue 220 has a flexible portion and a rigid or less-flexible portion.

In some embodiments, the cannula 110 may include a flexible portion. For example, the cannula 110 may include a laser-etched region to allow a user to bend the cannula 110. In some embodiments, flexibility in the cannula 110 may allow the user to direct the cannula 110 around a structure to reach a surgical site.

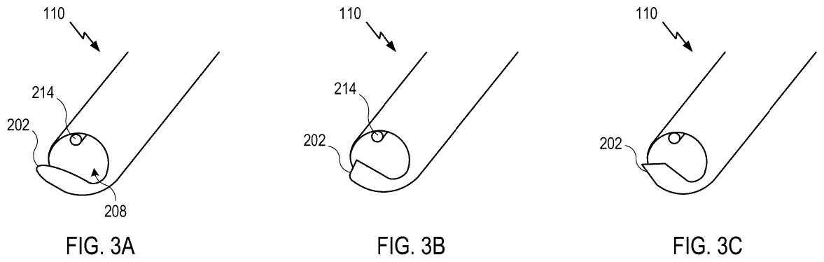

FIGS. 3A-C illustrate perspective views of example spatula geometries, in accordance with certain embodiments of the present disclosure. In the illustrated embodiment of FIG. 3A, the spatula 202 is shown with a rounded geometry. In some embodiments, having a rounded geometry reduces a chance of unintended tissue damage by spatula 202 at the surgical site.

FIG. 3B illustrates an example of a spatula 202 with a pointed geometry. In some embodiments, a pointed geometry in the spatula 202 allows for more precise lifting of material, such as a membrane on the retina, for removal thereof by laser light 216 from the optical fiber 214. For example, the spatula 202 may be positioned relative to the optical fiber 214 such that laser light emitted from the optical fiber 214 would impinge on the spatula 202 if uninterrupted.

FIG. 3C illustrates an example of spatula 202 with a flat edge. In some embodiments, different spatula 202 geometries may be more effective in engaging different materials (e.g., membranes) in different thicknesses, degrees of attachment, or having different adjoining structures.

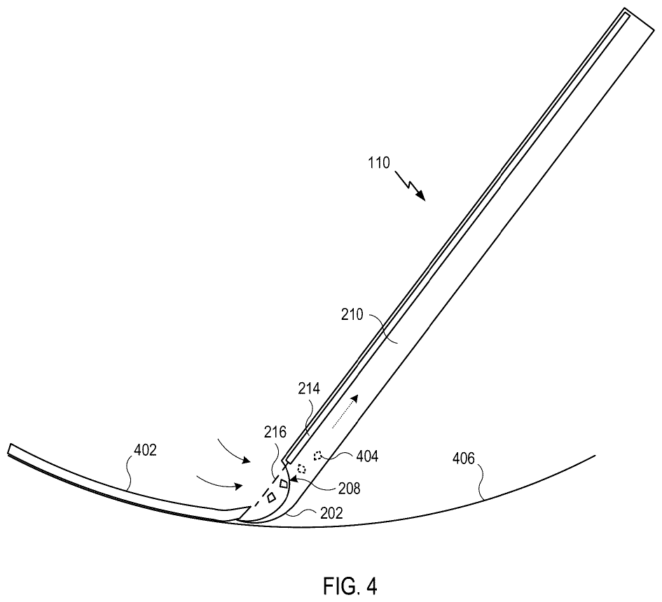

FIG. 4 illustrates a cross-sectional view of the cannula 110 engaged with a retinal membrane 402 within an eye of a patient, in accordance with certain embodiments of the present disclosure. In the illustrated example, the retinal membrane 402 may comprise, for example, an epiretinal membrane formed on the retina 406. As shown, the cannula 110 is positioned to remove the retinal membrane 402. The cannula 110 is configured to remove the retinal membrane 402 by engaging the retinal membrane with the spatula 202 to lift the retinal membrane 402 and breakdown the retinal membrane 402 with laser light 216 emitted from the optical fiber 214 within the interior 210 of the cannula 110. Fragments 404 of the retinal membrane 402, removed by the laser light 216, are aspirated into the cannula 110 via the aperture 208 for removal from the retina 406. In some embodiments, emission of the laser light 216 may be coordinated with negative pressure applied to the interior 210 of the cannula 110 to provide aspiration of the fragments 404 of the retinal membrane 402. For example, aspiration may be engaged prior to, at the same time as, or after firing of the laser light 216.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. A surgical instrument for membrane removal, the surgical instrument comprising:
a cannula;
a spatula formed at a distal end of the cannula, the spatula extending from the distal end of the cannula at a non-zero angle relative to an exterior surface of the cannula, the spatula at least partially defining an aperture at the distal end of the cannula; and
an optical fiber extending along the cannula, the optical fiber configured to direct laser light toward a surface of the spatula.

2. The surgical instrument of claim 1, wherein the non-zero angle of the spatula relative to the exterior surface of the cannula is between thirty and eighty degrees.

3. The surgical instrument of claim 1, wherein an outer edge of the spatula has a curved geometry.

4. The surgical instrument of claim 1, wherein an outer edge of the spatula has a pointed geometry.

5. The surgical instrument of claim 1, wherein the aperture is sized to allow for aspiration of material from a membrane into an interior of the cannula.

6. The surgical instrument of claim 1, wherein the cannula is at least partially flexible.

7. The surgical instrument of claim 1, further comprising an extendible tongue positioned within the cannula and extendible to reach a position outward from the spatula.

8. The surgical instrument of claim 1, wherein the proximal surface of the spatula is patterned to redirect the laser light from the optical fiber.

9. The surgical instrument of claim 1, wherein a distal surface of the spatula is patterned to facilitate traction against a membrane.

10. The surgical instrument of claim 1, wherein the laser light comprises a treatment laser light for severing ocular tissues.

11. The surgical instrument of claim 10, wherein the laser light further comprises a non-treatment laser light.

12. The surgical instrument of claim 11, wherein the spatula provides a backstop for the laser light.

13. The surgical instrument of claim 1, wherein the optical fiber extends along an interior of the cannula.

14. The surgical instrument of claim 1, wherein the optical fiber extends along at least a portion of an exterior of the cannula.

15. The surgical instrument of claim 1, further comprising a vacuum source in fluid communication with the cannula to facilitate aspiration of material through the aperture and into an interior of the cannula.
